(19) United States
(12) Patent Application Publication
(10) Pub. No.: US 2020/0281774 A1
(43) Pub. Date: Sep. 10, 2020

(54) ANIMAL BANDAGE WRAP INCLUDING AN ANTIMICROBIAL COATING

(71) Applicant: Parasol Medical, LLC, Buffalo Grove, IL (US)

(72) Inventor: Patrick E. Eddy, Allendale, MI (US)

(73) Assignee: Parasol Medical, LLC, Buffalo Grove, IL (US)

(21) Appl. No.: 16/293,311

(22) Filed: Mar. 5, 2019

(51) Int. Cl. A61F 13/00; A61F 13/06; A61F 13/02; A61K 31/14; A61K 47/10

(52) U.S. Cl. CPC A61F 13/00063; A61F 13/06; A61F 13/0273; A61F 2013/00285; A61K 47/10; A61F 2013/00119; A61F 2013/00217; A61K 31/14

(57) ABSTRACT

A wrap for a non-human animal comprises: a wrap for a non-human animal comprises: an elastic portion; an exterior surface; and an antimicrobial coating applied to the exterior surface. The antimicrobial coating includes a silane quaternary ammonium ion or salt thereof. The exterior surface can adhere to itself. The elastic portion can comprise natural rubber. The silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride. The antimicrobial coating applied to the exterior surface imparts antimicrobial properties onto the wrap. The antimicrobial coating can be applied to the wrap by applying a solution that includes the silane quaternary ammonium ion or salt thereof to the exterior surface. The silane quaternary ammonium ion or salt thereof can be between 0.1 percent and 10 percent by weight of the solution.

### BACKGROUND OF THE DISCLOSURE

An animal such as a dog or horse can become wounded. For example, the dog digging under a fence might cause the skin of the dog to develop an abrasion. As another example, a protruding nail in a stall might puncture the skin of a horse in the stall. Animals can develop skin wounds in many ways.

The wound can be treated to improve the likelihood of the skin healing properly. To treat the wound, a veterinarian or owner of the animal with the wound might clean the wound, apply an antiseptic to the wound, apply an antimicrobial ointment to the wound, apply a bandage, and wrap the bandage to secure the bandage to the skin of the animal. The veterinarian or owner can repeat the process until the skin has healed and the wound is gone.

Despite the treatment efforts, the wound might become infected. Bacteria and viruses can enter the wound and cause the infection. The bacteria and viruses can enter the wound from anywhere, including from the wrap used to wrap the bandage. Therefore, there is a need to prevent the wrap from being a source of bacteria and viruses that could enter into the wound and cause the infection.

In addition, wraps are used as an injury prevention tool for animals engaged in sporting activities. For example, an animal trainer, owner, or veterinarian might wrap the legs of a racing horse. Such wrapping of the legs of the horse is thought to help prevent a condition called “rundown,” where the fetlock of the leg hits the ground. The fetlock repeatedly hitting the ground can cause an abrasion wound. The wound can become infected. In any event, the wrap comes into contact with the skin of the animal, and the skin might have small wounds that are overlooked during wrapping. The wrap therefore might introduce bacteria and viruses into the overlooked wound. Therefore, there is a need again to prevent the wrap from being a source of bacteria and viruses that could enter into the wound and cause the infection, and to generally be free of bacteria and viruses to improve the health and well-being of the animal.

### BRIEF SUMMARY OF THE DISCLOSURE

According to a first aspect of the present disclosure, a wrap for a non-human animal comprises: an elastic portion; an exterior surface; and an antimicrobial coating applied to the exterior surface; wherein, the antimicrobial coating includes a silane quaternary ammonium ion or salt thereof.

Embodiments of the first aspect of the disclosure can include any one or a combination of the following features:
the exterior surface adheres to itself;
the elastic portion comprises natural rubber; and
the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

According to a second aspect of the present disclosure, a method of imparting antimicrobial properties onto a wrap for a non-human animal comprises: applying, to an exterior surface of a wrap for a non-human animal, the wrap including an elastic portion, a solution including a silane quaternary ammonium ion or salt thereof, and a solvent.

Embodiments of the second aspect of the disclosure can include any one or a combination of the following features:
the exterior surface adheres to itself;
the silane quaternary ammonium ion or salt thereof is between 0.1 percent and 10 percent by weight of the solution;
the solvent is isopropyl alcohol;
the isopropyl alcohol is between 30 percent to 90 percent by weight of the solution; and
the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

According to a third aspect of the present disclosure, a method of treating a non-human animal comprises: wrapping a portion of a body of a non-human animal with a wrap, the wrap including an elastic portion, an exterior surface, and an antimicrobial coating applied to the exterior surface; wherein, the antimicrobial coating includes a silane quaternary ammonium ion or salt thereof.

Embodiments of the third aspect of the disclosure can include any one or a combination of the following features:
the exterior surface adheres to itself during said wrapping;
the portion of the body of the non-human animal is a limb;
the non-human animal is a horse and the wrap is applied to help prevent a limb of the horse from experiencing rundown; and
the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

These and other features, advantages, and objects of the present disclosure will be further understood and appreciated by those skilled in the art by reference to the following specification, claims, and appended drawings.

### BRIEF DESCRIPTION OF THE DRAWINGS

FIG. 1 is an elevational view of a wrap for a non-human animal, illustrating an elastic portion with an exterior surface, onto which an antimicrobial coating is applied;

FIG. 2A is an overhead view of an alternative embodiment wrap for a non-human animal, illustrating a top layer and a portion of the top layer torn away to see the elastic portion, which itself is illustrated torn away to illustrate a bottom layer, the top layer having an exterior surface, onto which an antimicrobial coating is applied;

FIG. 2B is a cross-sectional elevational view taken along line IIB-IIB of FIG. 2, illustrating the elastic portion sandwiched between the top layer and the bottom layer, and an optional adhesive layer applied to the bottom layer;

FIG. 3A is a perspective view of the wrap of FIG. 1 beginning to be applied over the limb of a horse in order to treat the horse; and

FIG. 3B is a continuation of the scene presented in FIG. 3A, with the wrap having been applied over the limb of the horse, and the exterior surface adhering to itself without the use of an adhesive or tape.

### DETAILED DESCRIPTION

For purposes of description herein, it is to be understood that the specific devices and processes illustrated in the attached drawings, and described in the following specification are simply exemplary embodiments of the inventive concepts defined in the appended claims. Hence, physical characteristics relating to the embodiments disclosed herein are not to be considered as limiting, unless the claims expressly state otherwise.

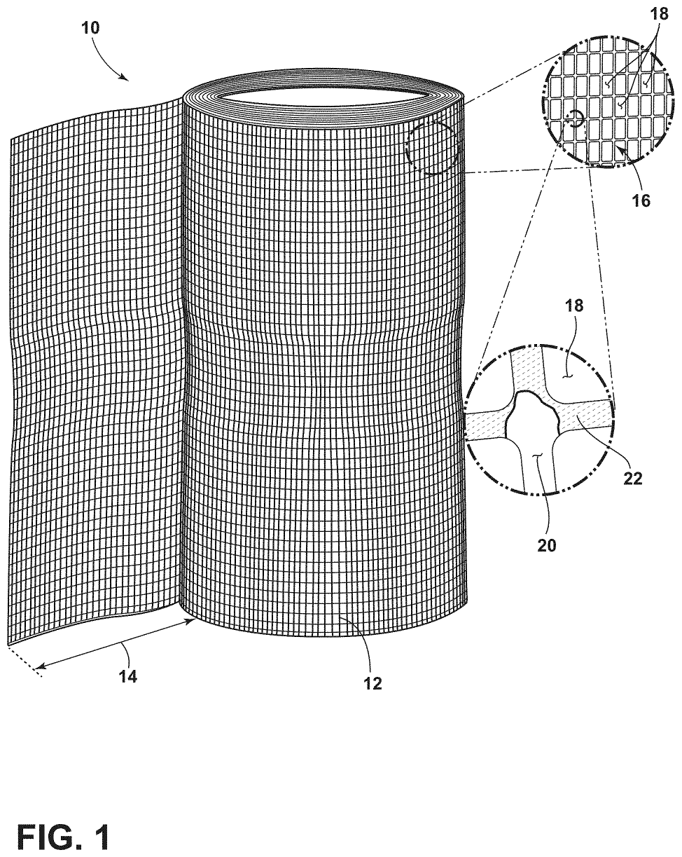

Referring to FIG. 1, an embodiment of a wrap 10 for a non-human animal is illustrated. The wrap 10 includes an elastic portion 12. Because the elastic portion 12 is elastic, the wrap 10 can be stretched at least along its length 14 and then contract. In the illustrated embodiment, the elastic portion 12 forms a web 16 surrounding voids 18, which makes the wrap 10 generally porous. The wrap 10 includes an exterior surface 20, which the elastic portion 12 can provide directly. The elastic portion 12 can comprise natural rubber, including 99 percent by weight or more natural rubber. Regardless of the composition of the elastic portion 12, the exterior surface 20 adheres to itself without the addition of an adhesive coating. In other words, the material (such as natural rubber) of the exterior surface 20 imparts a sufficient natural tackiness or other property to allow the wrap 10 not to release from itself when contacting itself when wrapped around the non-human animal. The wrap 10 further includes an antimicrobial coating 22 applied to the exterior surface 20. It is believed that an example of such a wrap 10, but not including the antimicrobial coating 22, is Vetrap Bandaging Tape (3M Company, Minnesota, USA).

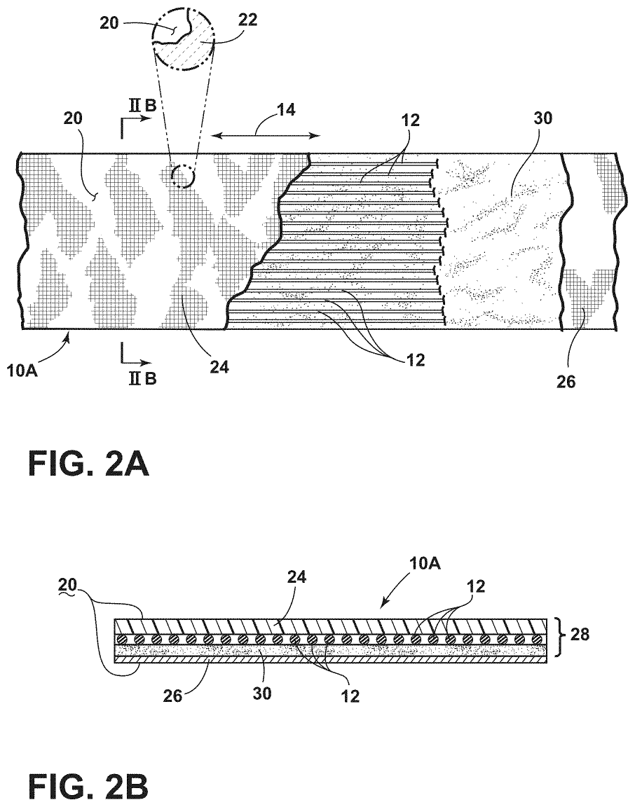

Referring now to FIGS. 2A and 2B, an alternative embodiment wrap 10A for a non-human animal is illustrated. The wrap 10A includes the elastic portion 12, which may take the form of strands of elastic material. Thus the wrap 10A can stretch along its length 14 and then contract. The wrap 10A further includes a top layer 24 and a bottom layer 26 sandwiching the elastic portion 12. The wrap 10A further includes a binder 28 that impregnates the elastic portion 12, the top layer 24, and the bottom layer 26. The wrap 10A can optionally include a pressure sensitive adhesive 30 coated onto the bottom layer 26. The top layer 24 can be a fabric. In some embodiments, the top layer 24 is a warp-knitted (weft insertion) fabric. The bottom layer 26 can be a fabric. In some embodiments, the bottom layer 26 is spun bond nonwoven nylon. In other embodiments, the bottom layer 26 is warp-knitted (weft insertion) fabric. The wrap 10A further includes the exterior surface 20, which may include the exposed surface of the top layer 26, the exposed surface of the bottom layer 26, or the exposed surface of the adhesive 30 if utilized, or any combination thereof. The wrap 10A further includes the antimicrobial coating 22 applied to the exterior surface 20. It is believed that an example of such a wrap 10A, but not including the antimicrobial coating 22, is PetFlex Flexible Cohesive Bandage (Andover Healthcare Inc., Massachusetts, USA).

The antimicrobial coating 22 includes a silane quaternary ammonium ion or salt thereof.

Preferred silane quaternary ammonium ions or salts thereof include 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride. Applying the antimicrobial coating 22 to the exterior surface 20 imparts antimicrobial properties on the exterior surface 20 and to the wrap 10, 10A generally, which prevents or lessens the ability of the exterior surface 20 to harbor harmful microbes, viruses, bacteria, and the like and spread them to the non-human animal on which the wrap 10, 10A is applied.

Further described herein is a novel method of imparting antimicrobial properties onto the wrap 10, 10A. The method comprises applying to the exterior surface 20 of the wrap 10, 10A, a solution including the silane quaternary ammonium ion or salt thereof, as described above. In addition to the silane quaternary ammonium ion or salt thereof, the solution can further include a solvent. A preferred solvent is isopropyl alcohol. After application of the solution to the exterior surface 20, the solvent, if utilized, generally evaporates, which leaves the silane quaternary ammonium ion or salt thereof applied to the exterior surface 20. Again, preferred silane quaternary ammonium ions or salts thereof include 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride. The solution can be applied to the exterior surface 20 via any application method, including electrostatic spraying, dipping, and coating.

The silane quaternary ammonium ion or salt thereof can comprise between 0.1 percent and 10 percent by weight of the solution. More preferably, the silane quaternary ammonium ion or salt thereof can comprise between 0.75 percent and 5 percent by weight of the solution. Even more preferably, the silane quaternary ammonium ion or salt thereof can comprise between 1.9 percent and 2.1 percent by weight of the solution.

As for the isopropyl alcohol, the isopropyl alcohol can comprise between 30 percent to 90 percent by weight of the solution. More preferably, the isopropyl alcohol can comprise between 55 percent and 65 percent by weight of the solution. An example preferable solution comprises (by weight) 60.0 percent isopropyl alcohol, 2.02 percent 3-(trimethoxysilyl)propyldimethyloctadecyl ammonium chloride, and 34.19 percent deionized water.

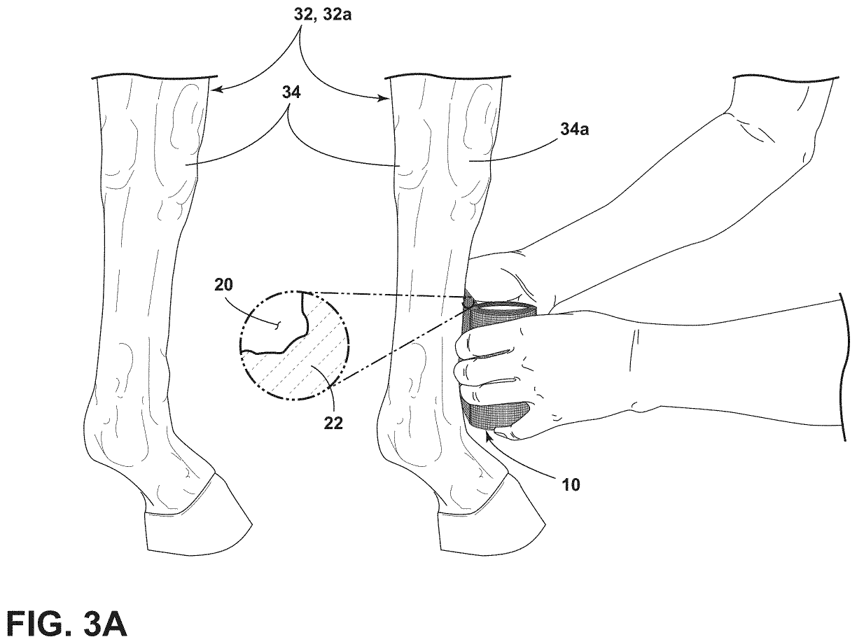

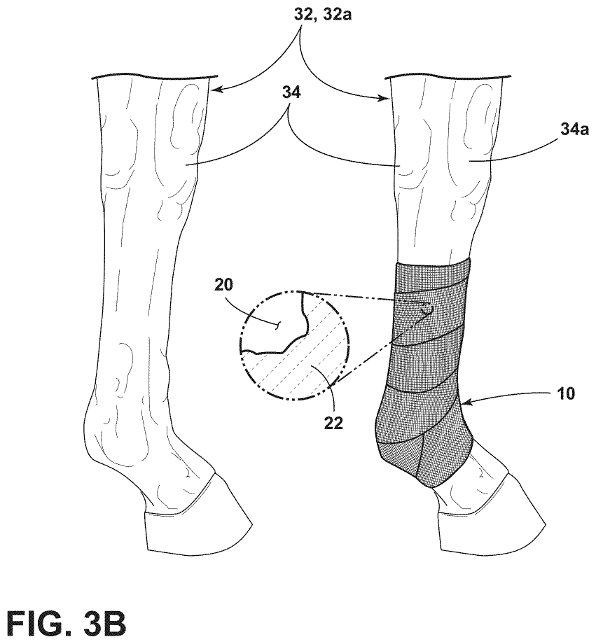

Referring now to FIGS. 3A and 3B, further described herein is a novel method of treating a non-human animal 32. The method includes wrapping a portion of a body 34 of the non-human animal 32 with an embodiment of the wrap 10, 10A. The wrap 10, 10A, as described above, includes the elastic portion 12, the exterior surface 20, and the antimicrobial coating 22 applied to the exterior surface 20. As discussed, the antimicrobial coating 22 includes a silane quaternary ammonium ion or salt thereof. The antimicrobial coating 22 can include any of the attributes described above. The wrap 10, 10A utilized can have any of the attributes described above, such as the exterior surface 20 adhering to itself during said wrapping. The self-adhering property avoids the need for a clip or excessive tape to keep the wrap 10, 10A applied to the portion of the body 34 of the non-human animal 32. The portion of the body 34 can be a limb 34a, among other things. Non-limiting examples of the non-human animal 32 include a horse 32a, a dog, and a cat. The wrap 10, 10A can be applied for various forms of treatment of the non-human animal 32, including to help prevent rundown of the horse 32a, or to aid in healing a wound. Because the antimicrobial coating 22 is applied to the exterior surface 20 of the wrap 10, 10A, the wrap 10, 10A is much less likely to harbor bacteria, viruses, and other microorganisms that could harm the non-human animal 32.

It is to be understood that variations and modifications can be made on the aforementioned structure without departing from the concepts of the present disclosure, and further it is to be understood that such concepts are intended to be covered by the following claims unless these claims by their language expressly state otherwise.

What is claimed is:

1. A wrap for a non-human animal comprising:
an elastic portion;
an exterior surface; and
an antimicrobial coating applied to the exterior surface;
wherein, the antimicrobial coating includes a silane quaternary ammonium ion or salt thereof.

2. The wrap of claim 1,
wherein, the exterior surface adheres to itself.

3. The wrap of claim 1,
wherein, the elastic portion comprises natural rubber.

4. The wrap of claim 1,
wherein, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

5. The wrap of claim 3,
wherein, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

6. A method of imparting antimicrobial properties onto a wrap for a non-human animal comprising:
applying, to an exterior surface of a wrap for a non-human animal, the wrap including an elastic portion, a solution including a silane quaternary ammonium ion or salt thereof and a solvent.

7. The method of claim 6,
wherein, the exterior surface adheres to itself.

8. The method of claim 6,
wherein, the silane quaternary ammonium ion or salt thereof is between 0.1 percent and 10 percent by weight of the solution.

9. The method of claim 6,
wherein, the solvent is isopropyl alcohol.

10. The method of claim 9,
wherein, the isopropyl alcohol is between 30 percent to 90 percent by weight of the solution.

11. The method of claim 6,
wherein, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

12. The method of claim 7,
wherein, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

13. The method of claim 8,
wherein, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

14. The method of claim 9,
wherein, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

15. The method of claim 10,
wherein, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

16. A method of treating a non-human animal comprising:
wrapping a portion of a body of a non-human animal with a wrap, the wrap including an elastic portion, an exterior surface, and an antimicrobial coating applied to the exterior surface;
wherein, the antimicrobial coating includes a silane quaternary ammonium ion or salt thereof.

17. The method of claim 16,
wherein, the exterior surface adheres to itself during said wrapping.

18. The method of claim 16,
wherein, the portion of the body of the non-human animal is a limb.

19. The method of claim 16,
wherein, the non-human animal is a horse and the wrap is applied to help prevent a limb of the horse from experiencing rundown.

20. The method of claim 16,
wherein, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.
